SEQUENCE LISTING (4 sequences)

SEQ ID NO: 1 - RNA, 20 nt, synthetic construct
  source 1..20: mol_type = other RNA
ccaccagcttacgcacaccg

SEQ ID NO: 2 - RNA, 20 nt, synthetic construct
  source 1..20: mol_type = other RNA
gggagccaacaccctcgctg

SEQ ID NO: 3 - RNA, 20 nt, synthetic construct
  source 1..20: mol_type = other RNA
gtagacgaaccggatgtcag

SEQ ID NO: 4 - RNA, 20 nt, synthetic construct
  source 1..20: mol_type = other RNA
cagccatgccttgtactcgg

(19) United States
(12) Patent Application Publication
(10) Pub. No.: US 2025/0258177 A1
(43) Pub. Date: Aug. 14, 2025

(54) MICROENVIRONMENTAL DETERMINANTS OF CLONAL HEMATOPOIESIS EXPANSION RATE

(71) Applicant: VANDERBILT UNIVERSITY, Nashville, TN (US)

(72) Inventors: Taralynn Mack, Nashville, TN (US); Alexander G. Bick, Nashville, TN (US)

(21) Appl. No.: 18/740,711

(22) Filed: Jun. 12, 2024

Related U.S. Application Data
(60) Provisional application No. 63/507,864, filed on Jun. 13, 2023.

(51) Int. Cl. G01N 33/574; C07K 16/18; C07K 16/26; C07K 16/30; C07K 16/40; C12N 9/22; C12N 15/11; G01N 33/68; G01N 33/74

(52) U.S. Cl. CPC G01N 33/57488; C07K 16/18; C07K 16/26; C07K 16/3015; C07K 16/40; C12N 9/226; C12N 15/11; G01N 33/57407; G01N 33/57426; G01N 33/6893; G01N 33/74; C12N 2310/20; G01N 2333/575; G01N 2333/91017; G01N 2800/2871; G01N 2800/323; G01N 2800/324; G01N 2800/50; G01N 2800/52

(57) ABSTRACT

Disclosed herein is a method for identifying a subject with increased risk of developing a cardiometabolic disease or a hematological cancer, that involves the steps of (a) assaying a blood, serum, or plasma sample from the subject for circulating levels of myeloid zinc finger 1 (MZF1), anti-müllerian hormone (AMH), TIMP metallopeptidase inhibitor 1 (TIMP1), glycine N-methyltransferase (GNMT), or a combination thereof; and (b) comparing the circulating levels of MZF1, AMH, TIMP1, GNMT, or a combination thereof, to control values, wherein presence of elevated circulating levels of MZF1, AMH, TIMP1, GNMT, or a combination thereof indicates an increased risk of developing a cardiometabolic disease and/or a hematological cancer.

### CROSS-REFERENCE TO RELATED APPLICATIONS

This claims benefit of U.S. Provisional Application No. 63/507,864, filed Jun. 13, 2023, which is hereby incorporated herein by reference in its entirety.

### SEQUENCE LISTING

This application contains a sequence listing filed in ST.26 format entitled “222230-1320 Sequence Listing” created on Jun. 12, 2024, and having 4,389 bytes. The content of the sequence listing is incorporated herein in its entirety.

### BACKGROUND OF THE INVENTION

Clonal hematopoiesis of indeterminate potential (CHIP) is a clonal expansion of blood cells caused by somatic mutations. CHIP confers risk of multiple aging diseases including blood cancer and cardiovascular disease. It is presently unknown what causes CHIP clones with identical driver mutations to expand at different rates in humans.

### SUMMARY OF THE INVENTION

Here, genetically predicted traits were leverage to identify factors that modify CHIP clonal growth rate. The passenger-approximated clonal expansion rate (PACER) method was used to quantify clonal growth rate for 4,370 individuals with CHIP mutations in the NHLBI TOPMed cohort. Polygenic risk scores (PRS) was calculated for DNA methylation clocks, inflammation related lab values, disease traits, and circulating protein levels. Associations between PRS and PACER score were tested with linear regression, controlling for covariates and correcting for multiple-hypothesis testing. CHIP clonal growth rate was significantly associated with both genetically predicted and measured methylation clocks (p<0.01). No associations were identified with any of the inflammation-related lab values, proteins or diseases. An unbiased proteome wide search identified circulating levels of myeloid zinc finger 1 (MZF1), anti-müllerian hormone (AMH), TIMP metallopeptidase inhibitor 1 (TIMP1), glycine N-methyltransferase (GNMT) as altering CHIP clonal expansion rate (p<4.1×10⁻⁶). An aging microenvironment was associated with increased CHIP expansion rate as well as four specific circulating micro-environment proteins. Contrary to prior murine models, inflammation does not appear to be a root cause of CHIP clonal expansion rate in humans.

Therefore, disclosed herein is a method for identifying a subject with increased risk of developing a cardiometabolic disease or a hematological cancer, that involves the steps of (a) assaying a blood, serum, or plasma sample from the subject for circulating levels of myeloid zinc finger 1 (MZF1), anti-müllerian hormone (AMH), TIMP metallopeptidase inhibitor 1 (TIMP1), glycine N-methyltransferase (GNMT), or a combination thereof; and (b) comparing the circulating levels of MZF1, AMH, TIMP1, GNMT, or a combination thereof, to control values, wherein presence of elevated circulating levels of MZF1, AMH, TIMP1, GNMT, or a combination thereof indicates an increased risk of developing a cardiometabolic disease and/or a hematological cancer.

In some embodiments, the cardiometabolic disease is atherosclerosis, coronary heart disease (CHD) or ischemic stroke (IS). In some embodiments, the hematological cancer is a leukemia, a lymphoma, a myeloma or a blood syndrome. In some embodiments, the leukemia is acute myeloid leukemia (AML) or chronic myelogenous leukemia (CML). In some embodiments, the blood syndrome is myelodysplastic syndrome (MDS). In some embodiments, the subjects exhibits one or more risk factors of being a smoker, having a high level of total cholesterol or having high level of high-density lipoprotein (HDL).

In some embodiments, the method further involves administering to the subject an inhibitor of MZF1, AMH, TIMP1, GNMT, or a combination thereof. In some embodiments, the inhibitor of MZF1, AMH, TIMP1, GNMT, or a combination thereof is an antibody. In some embodiments, the inhibitor of MZF1, AMH, TIMP1, GNMT, or a combination thereof is an small molecule. In some embodiments, the inhibitor of MZF1, AMH, TIMP1, GNMT, or a combination thereof is polynucleotide encoding a gRNA and Cas gene.

Also disclosed is a method for treating a cardiometabolic disease or a hematological cancer, that involves administering to the subject an inhibitor of MZF1, AMH, TIMP1, GNMT, or a combination thereof. In some embodiments, the inhibitor of MZF1, AMH, TIMP1, GNMT, or a combination thereof is an antibody. In some embodiments, the inhibitor of MZF1, AMH, TIMP1, GNMT, or a combination thereof is an small molecule. In some embodiments, the inhibitor of MZF1, AMH, TIMP1, GNMT, or a combination thereof is polynucleotide encoding a gRNA and Cas gene.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 is a schematic representation of the study design. Polygenic risk scores were calculated for traits of interest for individuals in the TOPMed cohort with CHIP mutations using either PRScsx, PRScs, or PLINK. These scores were then regressed with clonal expansion rate (estimated using PACER) to determine if traits were associated with CHIP progression.

FIGS. 2A and 2B show regressions of clonal expansion rate with DNA methylation aging measures. FIG. 2A shows associations between clonal expansion rate and measured DNAm aging measures (n=297) stratified by CHIP gene mutation. FIG. 2B shows associations between clonal expansion rate and DNAm aging measure PRS (n=4,370) stratified by CHIP gene mutation.

FIGS. 3A to 3C show regressions of clonal expansion rate with inflammation. Only p-values>0.05 are shown. FIG. 3A is a heat map showing associations between clonal expansion rate and PRS for inflammation-related proteins from the Somalogic database stratified by CHIP gene. Significance and direction of effect are denoted by the color of the box. FIG. 3B shows a subset of inflammation-related proteins that have a known pro/anti-inflammatory effect. Direction of effect is denoted by color. FIG. 3C is a heat map showing associations between clonal expansion and PRS for inflammation-related phenotypes. Significance and direction of effect are denoted by the color of the box.

FIGS. 4A to 4C show regressions of clonal expansion rate with predicted protein levels. FIG. 4A is a volcano plot showing significantly associated protein PRS from the Somalogic database. Colored points are statistically significant. The color of the point shows the direction of the effect (blue=negative, red=positive). FIG. 4B is a forest plot showing the CHIP gene mutation specific correlations for the significant hits. FIG. 4C contains Gene Set Enrichment Analysis (GSEA) results showing the pathways implicated in the associations between clonal expansion rate and circulating protein PRS.

FIGS. 5A to 5C show correlation in DNA methylation aging measures. FIG. 5A shows is a correlation matrix within measured epigenetic aging data. FIG. 5B is a correlation matrix within predicted epigenetic aging data. FIG. 5C is a box plot showing correlation between calculated PRS (grouped into quartiles) and z-scored measured methylation aging data.

FIGS. 6A and 6B shows regressions of clonal expansion rate with inflammation (full results). FIG. 6A is a heat map showing associations between clonal expansion rate and PRS for inflammation-related proteins from the Somalogic database stratified by CHIP gene. Significance and direction of effect are denoted by the color of the box. FIG. 6B is a heat map showing associations between clonal expansion and PRS for inflammation-related phenotypes. Significance and direction of effect are denoted by the color of the box.

FIGS. 7A and 7B shows regressions of clonal expansion rate with predicted protein levels in CHIP gene-specific analyses. Volcano plots showing significantly associated protein PRS from the Somalogic database. Colored points are statistically significant. The color of the point shows the direction of the effect (blue=negative, red=positive). FIG. 7A shows DNMT3A-specific CHIP, and FIG. 7B shows TET2-specific CHIP.

FIG. 8 shows sex-stratified regressions of clonal expansion rate with predicted protein levels in overall CHIP.

FIGS. 9A and 9B show hallmark pathways in Gene Set Enrichment Analysis (GSEA). FIG. 9A is a heat map showing the pathways most implicated in both overall CHIP and gene-specific CHIP. Color denotes the direction of the association. FIG. 9B shows protein PRSs contributing to the hallmark pathways most strongly associated with clonal expansion rate. Color denotes the direction of the association.

FIGS. 10A to 10C shows Gene Set Enrichment Analysis (GSEA) results for CHIP gene-specific analyses. FIG. 10A shows pathways implicated in the associations between clonal expansion rate and circulating protein PRS in DNMT3A-specific CHIP. FIG. 10B shows TET2-specific CHIP. FIG. 10C shows correlation between pathways implicated in DNMT3A & TET2-specific CHIP.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Gene expression” or alternatively a “gene product” refers to the nucleic acids or amino acids (e.g., peptide or polypeptide) generated when a gene is transcribed and translated.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non cancerous cell or tissue) is undetectable.

A high expression level of the gene can occur because of over expression of the gene or an increase in gene copy number. The gene can also be translated into increased protein levels because of deregulation or absence of a negative regulator. Lastly, high expression of the gene can occur due to increased stabilization or reduced degradation of the protein, resulting in accumulation of the protein.

A “gene expression profile” or “gene signature” refers to a pattern of expression of at least one biomarker that recurs in multiple samples and reflects a property shared by those samples, such as mutation, response to a particular treatment, or activation of a particular biological process or pathway in the cells. A gene expression profile differentiates between samples that share that common property and those that do not with better accuracy than would likely be achieved by assigning the samples to the two groups at random. A gene expression profile may be used to predict whether samples of unknown status share that common property or not. Some variation between the biomarker(s) and the typical profile is to be expected, but the overall similarity of biomarker(s) to the typical profile is such that it is statistically unlikely that the similarity would be observed by chance in samples not sharing the common property that the biomarker(s) reflects.

### Immunoassays

Methods for detecting and/or measuring expression of proteins, such as MZF1, AMH, TIMP1, or GNMT, are known in the art.

Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

### MZF1, AMH, TIMP1, GNMT Inhibitors

The term “inhibitor” refers to any compound capable of inhibiting the production, level, activity, expression or presence of MZF1, AMH, TIMP1, or GNMT. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the MZF1, AMH, TIMP1, GNMT protein, either by ATP-competitive inhibition of the active site, allosteric modulation of the protein structure, disruption of protein-protein interactions, or by inhibiting the transcription, translation, post-translational modification, or stability of MZF1, AMH, TIMP1, GNMT protein.

### Antibodies

The term “antibody” and the like as used herein refers to a whole antibody or a fragment thereof that interacts with (e.g., by binding, steric hindrance, stabilizing/destabilizing, spatial distribution) a MZF1, AMH, TIMP1, or GNMT epitope. A naturally occurring IgG “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “epitope binding domain” or “EBD” refers to portions of a binding molecule (e.g., an antibody or epitope-binding fragment or derivative thereof), that specifically interacts with (e.g., by binding, steric hindrance, stabilizing/destabilizing, spatial distribution) a binding site on a target epitope. EBD also refers to one or more fragments of an antibody that retain the ability to specifically interact with (e.g., by binding, steric hindrance, stabilizing/destabilizing, spatial distribution) a PRMT5 epitope and inhibit signal transduction. Examples of antibody fragments include, but are not limited to, an scFv, a Fab fragment, a monovalent fragment consisting of the VL, VH, CL and CH1 domains; a F (ab).sub.2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).

Such single chain antibodies are also intended to be encompassed within the terms “fragment”, “epitope-binding fragment” or “antibody fragment”. These fragments are obtained using conventional techniques known to those of skill in the art, and the fragments are screened for utility in the same manner as are intact antibodies.

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8:1057-1062; and U.S. Pat. No. 5,641,870), and also include Fab fragments, F(ab′) fragments, and anti-idiotypic (anti-Id) antibodies (including, e.g., anti-Id antibodies to antibodies of the invention), and epitope-binding fragments of any of the above.

EBDs also include single domain antibodies, maxibodies, unibodies, minibodies, triabodies, tetrabodies, v-NAR and bis-scFv, as is known in the art (see, e.g., Hollinger and Hudson, (2005) Nature Biotechnology 23:1126-1136), bispecific single chain diabodies, or single chain diabodies designed to bind two distinct epitopes. EBDs also include antibody-like molecules or antibody mimetics, which include, but not limited to minibodies, maxybodies, Fn3 based protein scaffolds, Ankrin repeats (also known as DARpins), VASP polypeptides, Avian pancreatic polypeptide (aPP), Tetranectin, Affililin, Knottins, SH3 domains, PDZ domains, Tendamistat, Neocarzinostatin, Protein A domains, Lipocalins, Transferrin, and Kunitz domains that specifically bind epitopes, which are within the scope of the invention. Antibody fragments can be grafted into scaffolds based on polypeptides such as Fibronectin type III (Fn3) (see U.S. Pat. No. 6,703,199, which describes fibronectin polypeptide monobodies).

The present invention also encompasses an antibody to PRMT5, which is an isolated antibody, monovalent antibody, bivalent antibody, multivalent antibody, bivalent antibody, biparatopic antibody, bispecific antibody, monoclonal antibody, human antibody, recombinant human antibody, or any other type of antibody or epitope-binding fragment or derivative thereof.

The phrase “isolated antibody”, as used herein, refers to antibody that is substantially free of other antibodies having different antigenic specificities (e.g., an isolated antibody that specifically binds PRMT5 is substantially free of antibodies that specifically bind antigens other than PRMT5). An isolated antibody that specifically binds PRMT5 may, however, have cross-reactivity to other antigens, such as PRMT5 molecules from other species. Moreover, an isolated antibody may be substantially free of other cellular material and/or chemicals.

The term “monovalent antibody” as used herein, refers to an antibody that binds to a single epitope on a target molecule such as PRMT5.

The term “bivalent antibody” as used herein, refers to an antibody that binds to two epitopes on at least two identical PRMT5 target molecules. The bivalent antibody may also crosslink the target PRMT5 molecules to one another. A “bivalent antibody” also refers to an antibody that binds to two different epitopes on at least two identical PRMT5 target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for the PRMT5, the multivalent antibody (e.g., a PRMT5 biparatopic antibody) has a binding moiety for two domains of PRMT5, respectively.

The multivalent antibody mediates biological effect or which modulates a disease or disorder in a subject (e.g., by mediating or promoting cell killing, or by modulating the amount of a substance which is bioavailable.

The term “multivalent antibody” also refers to a single binding molecule that has more than one antigen-binding moieties for two separate WRM target molecules. For example, an antibody that binds to both a PRMT5 target molecule and a second target molecule that is not PRMT5. In one embodiment, a multivalent antibody is a tetravalent antibody that has four epitope binding domains. A tetravalent molecule may be bispecific and bivalent for each binding site on that target molecule.

The term “biparatopic antibody” as used herein, refers to an antibody that binds to two different epitopes on a single PRMT5 target. The term also includes an antibody, which binds to two domains of at least two PRMT5 targets, e.g., a tetravalent biparatopic antibody.

The term “bispecific antibody” as used herein, refers to an antibody that binds to two or more different epitopes on at least two different targets (e.g., a PRMT5 and a target that is not PRMT5).

The phrases “monoclonal antibody” or “monoclonal antibody composition” as used herein refers to polypeptides, including antibodies, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The phrase “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding site” as used herein comprises an area on a PRMT5 target molecule to which an antibody or antigen binding fragment selectively binds.

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics.

Generally, antibodies specific for a particular target antigen will bind to an epitope on the target antigen in a complex mixture of proteins and/or macromolecules.

As used herein, the term “Affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a K_{D} of 10⁻⁸ M or less, 10⁻⁹M or less, or 10⁻¹⁰ M, or 10⁻¹¹ M or less, or 10⁻¹² M or less, or 10⁻¹³ M or less for a target antigen. However, high affinity binding can 10 vary for other antibody isotypes. For example, high affinity binding for an IgM isotype refers to an antibody having a K_{D} of 10⁻⁷M or less, or 10⁻⁸ M or less.

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

An inhibitory antibody can be prepared; alternatively, many PRMT5 antibodies are known in the art. Any inhibitory antibody or fragment thereof can be used with any method disclosed herein.

### Guide RNA

In some embodiments, the inhibitor of MZF1, AMH, TIMP1, GNMT, or a combination thereof is polynucleotide encoding a gRNA and Cas gene. A gRNA, as used herein, refers to a nucleic acid that promotes the specific targeting or homing of a gRNA molecule/Cas9 molecule complex to a target nucleic acid. gRNA molecules can be unimolecular (having a single RNA molecule), sometimes referred to herein as “chimeric” gRNAs, or modular (comprising more than one, and typically two, separate RNA molecules). gRNA are a synthetic fusion of the endogenous bacterial crRNA and tracrRNA. gRNA provide both targeting specificity and scaffolding/binding ability for Cas9 nuclease. They do not exist in nature. gRNA are sometimes referred to as “single guide RNA” or “sgRNA”. A gRNA molecule comprises a number of domains, which are described in more detail below.

In some embodiments the inhibitor of MZF1 is polynucleotide encoding a gRNA and Cas gene. An example gRNA for MZF1 is CCACCAGCTTACGCACACCG (SEQ ID NO:1).

In some embodiments the inhibitor of AMH is polynucleotide encoding a gRNA and Cas gene. An example gRNA for AMH is GGGAGCCAACACCCTCGCTG (SEQ ID NO:2).

In some embodiments the inhibitor of TIMP1 is polynucleotide encoding a gRNA and Cas gene. An example gRNA for TIMP1 is GTAGACGAACCGGATGTCAG (SEQ ID NO:3).

In some embodiments the inhibitor of GNMT is polynucleotide encoding a gRNA and Cas gene. An example gRNA for GNMT is CAGCCATGCCTTGTACTCGG (SEQ ID NO:4).

In some embodiments, the gRNA comprises, preferably from 5′ to 3′: a targeting domain (which is complementary to a target nucleic acid); a first complementarity domain; a linking domain; a second complementarity domain (which is complementary to the first complementarity domain); a proximal domain; and optionally, a tail domain.

In some embodiments, the targeting domain comprises a nucleotide sequence that is complementary, e.g., at least 80%, 85%, 90%, 95%, or 100% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises, in the 5′ to 3′ direction, an optional secondary domain, and a core domain. In some embodiments, the core domain is fully complementary with the target sequence. In some embodiments, the targeting domain is 5 to 50, 10 to 40, e.g., 10 to 30, e.g., 15 to 30, e.g., 15 to 25 nucleotides in length. In some embodiments, the targeting domain is 15, 16, 17, 18, 19, 20, 21, 22, 23, 24 or 25 nucleotides in length. The strand of the target nucleic acid with which the targeting domain is complementary is referred to herein as the complementary strand. Some or all of the nucleotides of the domain can have a modification.

The first complementarity domain is complementary with the second complementarity domain, and In some embodiments, has sufficient complementarity to the second complementarity domain to form a duplexed region under at least some physiological conditions. In some embodiments, the first complementarity domain is 5 to 30 nucleotides in length. In some embodiments, the first complementarity domain is 5 to 25 nucleotides in length. In some embodiments, the first complementary domain is 7 to 25 nucleotides in length. In some embodiments, the first complementary domain is 7 to 22 nucleotides in length. In some embodiments, the first complementary domain is 7 to 18 nucleotides in length. In some embodiments, the first complementary domain is 7 to 15 nucleotides in length. In some embodiments, the first complementary domain is 5, 6, 7, 8, 9, 10, 11, 12, 13, 14, 15, 16, 17, 18, 19, 20, 21, 22, 23, 24, or 25 nucleotides in length.

In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4-9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

The first complementarity domain can share homology with, or be derived from, a naturally occurring first complementarity domain. In some embodiments, it has at least 50% homology with a first complementarity domain disclosed herein, e.g., a Streptococcus pyogenes (S. pyogenes) or Streptococcus thermophiles (S. thermophiles), first complementarity domain.

A linking domain serves to link the first complementarity domain with the second complementarity domain of a unimolecular gRNA. The linking domain can link the first and second complementarity domains covalently or non-covalently. In some embodiments, the linkage is covalent. In some embodiments, the linking domain covalently couples the first and second complementarity domains. In some embodiments, the linking domain is, or comprises, a covalent bond interposed between the first complementarity domain and the second complementarity domain. Typically, the linking domain comprises one or more, e.g., 2, 3, 4, 5, 6, 7, 8, 9, or 10 nucleotides.

In modular gRNA molecules the two molecules can be associated by virtue of the hybridization of the complementarity domains.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length.

In some embodiments, a linking domain is 2, 3, 4, 5, 6, 7, 8, 9, 10, 15, 20, or 25 or more nucleotides in length. In some embodiments, a linking domain is 2 to 50, 2 to 40, 2 to 30, 2 to 20, 2 to 10, or 2 to 5 nucleotides in length. In some embodiments, a linking domain shares homology with, or is derived from, a naturally occurring sequence, e.g., the sequence of a tracrRNA that is 5′ to the second complementarity domain. In some embodiments, the linking domain has at least 50% homology with a linking domain disclosed herein.

In some embodiments, a modular gRNA can comprise additional sequence, 5′ to the second complementarity domain, referred to herein as the 5′ extension domain. In some embodiments, the 5′ extension domain is, 2-10, 2-9, 2-8, 2-7, 2-6, 2-5, 2-4 nucleotides in length. In some embodiments, the 5′ extension domain is 2, 3, 4, 5, 6, 7, 8, 9, or 10 or more nucleotides in length.

The second complementarity domain is complementary with the first complementarity domain, and In some embodiments, has sufficient complementarity to the second complementarity domain to form a duplexed region under at least some physiological conditions. In some embodiments, the second complementarity domain can include sequence that lacks complementarity with the first complementarity domain, e.g., sequence that loops out from the duplexed region.

In some embodiments, the second complementarity domain is 5 to 27 nucleotides in length. In some embodiments, it is longer than the first complementarity region.

In some embodiments, the second complementary domain is 7 to 27 nucleotides in length. In some embodiments, the second complementary domain is 7 to 25 nucleotides in length. In some embodiments, the second complementary domain is 7 to 20 nucleotides in length. In some embodiments, the second complementary domain is 7 to 17 nucleotides in length. In some embodiments, the complementary domain is 5, 6, 7, 8, 9, 10, 11, 12, 13, 14, 15, 16, 17, 18, 19, 20, 21, 22, 23, 24 or 25 nucleotides in length.

In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length.

In some embodiments, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In some embodiments, it has at least 50% homology with a second complementarity domain disclosed herein, e.g., an S. pyogenes, or S. thermophilus, first complementarity domain.

Some or all of the nucleotides of the domain can have a modification.

In some embodiments, the proximal domain is 5 to 20 nucleotides in length. In some embodiments, the proximal domain can share homology with or be derived from a naturally occurring proximal domain. In some embodiments, it has at least 50% homology with a proximal domain disclosed herein, e.g., an S. pyogenes, or S. thermophilus, proximal domain.

A broad spectrum of tail domains are suitable for use in gRNA molecules. In some embodiments, the tail domain is 0 (absent), 1, 2, 3, 4, 5, 6, 7, 8, 9, or 10 nucleotides in length. In some embodiments, the tail domain nucleotides are from or share homology with sequence from the 5′ end of a naturally occurring tail domain. In some embodiments, the tail domain includes sequences that are complementary to each other and which, under at least some physiological conditions, form a duplexed region.

In some embodiments, the tail domain is absent or is 1 to 50 nucleotides in length. In some embodiments, the tail domain can share homology with or be derived from a naturally occurring proximal tail domain. In some embodiments, it has at least 50% homology with a tail domain disclosed herein, e.g., an S. pyogenes, or S. thermophilus, tail domain.

In some embodiments, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

Methods for Designing gRNAs

Methods for selection and validation of target sequences as well as off-target analyses are described, e.g., in. Mali et al., 2013 SCIENCE 339 (6121): 823-826; Hsu et al., 2013 NAT BIOTECHNOL, 31 (9): 827-32; Fu et al., 2014 NAT BIOTECHNOL, doi: 10.1038/nbt.2808. PubMed PMID: 24463574; Heigwer et al., 2014 NAT METHODS 11 (2): 122-3. doi: 10.1038/nmeth.2812. PubMed PMID: 24481216; Bae et al., 2014 BIOINFORMATICS PubMed PMID: 24463181; Xiao A et al., 2014 BIOINFORMATICS PubMed PMID: 24389662.

For example, a software tool can be used to optimize the choice of sgRNA within a user's target sequence, e.g., to minimize total off-target activity across the genome. Off target activity may be other than cleavage. For each possible gRNA choice e.g., using S. pyogenes Cas9, the tool can identify all off-target sequences (e.g., preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate gRNA molecules can be evaluated by art-known methods.

### Cas9 Molecules

Cas9 molecules of a variety of species can be used in the methods and compositions described herein. While the S. pyogenes and S. thermophilus Cas9 molecules are typically used, Cas9 molecules of, derived from, or based on the Cas9 proteins of other species can be used, e.g., Staphylococcus aureus, Neisseria meningitides.

A Cas9 molecule, as that term is used herein, refers to a molecule that can interact with a sgRNA molecule and, in concert with the sgRNA molecule, localize (e.g., target or home) to a site which comprises a target domain and PAM sequence.

In some embodiments, the Cas9 molecule is capable of cleaving a target nucleic acid molecule. Exemplary naturally occurring Cas9 molecules are described in Chylinski et al., RNA Biology 2013; 10:5, 727-737. Naturally occurring Cas9 molecules possess a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In some embodiments, a Cas9 molecules can include all or a subset of these properties. In typical embodiments, Cas9 molecules have the ability to interact with a gRNA molecule and, in concert with the gRNA molecule, localize to a site in a nucleic acid. Other activities, e.g., PAM specificity, cleavage activity, or helicase activity can vary more widely in Cas9 molecules.

Cas9 molecules with desired properties can be made in a number of ways, e.g., by alteration of a parental, e.g., naturally occurring Cas9 molecules to provide an altered Cas9 molecule having a desired property. For example, one or more mutations or differences relative to a parental Cas9 molecule can be introduced. Such mutations and differences comprise: substitutions (e.g., conservative substitutions or substitutions of non-essential amino acids); insertions; or deletions. In some embodiments, a Cas9 molecule can comprises one or more mutations or differences, e.g., at least 1, 2, 3, 4, 5, 10, 15, 20, 30, 40 or 50 mutations but less than 200, 100, or 80 mutations relative to a reference Cas9 molecule.

In some embodiments, a mutation or mutations do not have a substantial effect on a Cas9 activity, e.g. a Cas9 activity described herein. In some embodiments, a mutation or mutations have a substantial effect on a Cas9 activity, e.g. a Cas9 activity described herein. In some embodiments, exemplary activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in an N-terminal RuvC-like domain. In some embodiments, a mutation(s) is present in an HNH-like domain. In some embodiments, mutations are present in both an N-terminal RuvC-like domain and an HNH-like domain.

Whether or not a particular sequence, e.g., a substitution, may affect one or more activity, such as targeting activity, cleavage activity, etc, can be evaluated or predicted, e.g., by evaluating whether the mutation is conservative. In some embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

Naturally occurring Cas9 molecules can recognize specific PAM sequences, for example the PAM recognition sequences for S. pyogenes, S. thermophilus, S. mutans, S. aureus and N. meningitidis.

In some embodiments, a Cas9 molecule has the same PAM specificities as a naturally occurring Cas9 molecule. In other embodiments, a Cas9 molecule has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule recognizes to decrease off target sites and/or improve specificity; or eliminate a PAM recognition requirement. In some embodiments, a Cas9 molecule can be altered, e.g., to increase length of PAM recognition sequence and/or improve Cas9 specificity to high level of identity to decrease off target sites and increase specificity. In some embodiments, the length of the PAM recognition sequence is at least 4, 5, 6, 7, 8, 9, 10 or 15 amino acids in length. Cas9 molecules that recognize different PAM sequences and/or have reduced off-target activity can be generated using directed evolution.

In some embodiments, a Cas9 molecule comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

In some embodiments, the Cas9 is a “high fidelity” spCas9 variants (HF-Cas9), such as those designed according to principles disclosed by Joung and colleagues (Kleinstiver, et al., 2016, which is incorporated herein in its entirety).

### Silencing Oligonucleotides

In some embodiments, the inhibitor is an siRNA (short inhibitory RNA), shRNA (short or small hairpin RNA), IRNA (interference RNA) agent, RNAi (RNA interference) agent, dsRNA (double-stranded RNA), microRNA, and the like, which specifically binds to the MZF1, AMH, TIMP1, or GNMT mRNA and which mediates the targeted cleavage of the RNA transcript via an RNA-induced silencing complex (RISC) pathway. In one embodiment, the inhibitor is an oligonucleotide composition that activates the RISC complex/pathway. In another embodiment, the RNAi agent comprises an antisense strand sequence (antisense oligonucleotide). In one embodiment, the RNAi comprises a single strand. This single-stranded RNAi agent oligonucleotide or polynucleotide can comprise the sense or antisense strand, as described by Sioud 2005 J. Mol. Bioi. 348:1079-1090, and references therein. Thus the disclosure encompasses RNAi agents with a single strand comprising either the sense or antisense strand of an RNAi agent described herein. The use of the RNAi agent to MZF1, AMH, TIMP1, or GNMT results in a decrease of MZF1, AMH, TIMP1, or GNMT post-translational modification, production, expression, level, stability and/or activity, e.g., a “knock-down” or “knock-out” of the MZF1, AMH, TIMP1, or GNMT target gene or target sequence.

RNA interference is a post-transcriptional, targeted gene-silencing technique that, usually, uses double-stranded RNA (dsRNA) to degrade messenger RNA (mRNA) containing the same sequence as the dsRNA. The process of RNAi occurs naturally when ribonuclease III (Dicer) cleaves longer dsRNA into shorter fragments called siRNAs. Naturally-occurring siRNAs (small interfering RNAs) are typically about 21 to 23 nucleotides long and comprise about 19 base pair duplexes. The smaller RNA segments then mediate the degradation of the target mRNA. Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex.

“RNAi” (RNA interference) has been studied in a variety of systems. Early work in Drosophila embryonic lysates (Elbashir et al. 2001 EMBO J. 20:6877 and Tuschl et al. International PCT Publication No. WO 01/75164) revealed certain parameters for siRNA length, structure, chemical composition, and sequence that are beneficial to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of an siRNA duplex is usually required for siRNA activity. Later work showed that a 3′-terminal dinucleotide overhang can be replaced by a 3′ end cap, provided that the 3′ end cap still allows the molecule to mediate RNA interference; the 3′ end cap also reduces sensitivity of the molecule to nucleases. See, for example, U.S. Pat. Nos. 8,097,716; 8,084,600; 8,404,831; 8,404,832; and 8,344,128, and International Patent Application PCT/US14/58705. Additional later work on artificial RNAi agents showed that the strand length could be shortened, or a single-stranded nick could be introduced into a strand. International Patent Applications PCT/US14/58703 and PCT/US14/59301. In addition, mismatches can be introduced between the sense and anti-sense strands and a variety of modifications can be used. Any of the these and various other formats for RNAi agents known in the art can be used to produce RNAi agents to PRMT5.

In various embodiments, the RNAi agent can comprise nucleotides (e.g., RNA or DNA), modified nucleotides, and/or nucleotide substitutes. In some embodiments, the RNAi agent can comprise RNA. In some embodiments, the RNAi agent can comprise RNA, with several of the RNA nucleotides replaced with DNA or a modified nucleotide. In various embodiments, the nucleotide (consisting of a phosphate, sugar and base) can be modified and/or substituted at the phosphate, sugar and/or base. For example, the sugar can be modified at the 2′ carbon, as is known in the art. In another non-limiting example, the phosphate can be modified or replaced, e.g., substituted with a modified internucleoside linker.

In some embodiments, the RNAi agent comprises an 18-mer strand terminating in a 3′ phosphate or modified internucleoside linker, and further comprising a spacer (but no phosphate or modified internucleoside linker, or 3′ end cap). Thus: In some embodiments, the RNAi agent comprises an 18-mer strand terminating in a 3′ phosphate or modified internucleoside linker, and further comprising a spacer (e.g., ribitol). In some embodiments, the RNAi comprises an 18-mer strand terminating in a 3′ phosphate or modified internucleoside linker, and further comprising a spacer (e.g., a ribitol). In some embodiments, the RNAi comprises an 18-mer strand terminating in a 3′ phosphate or modified internucleoside linker, and further comprising, in 5′ to 3′ order, a spacer (e.g., a ribitol), a second phosphate or modified internucleoside linker, and a second spacer (e.g., ribitol).

In various embodiments, one or both strands can comprise ribonucleotide subunits, or one or more nucleotide can optionally be modified or substituted. Thus, in various embodiments, the RNAi agent can either contain only naturally-occurring ribonucleotide subunits, or one or more modifications to the sugar, phosphate or base of one or more of nucleotide subunits. In one embodiment, the modifications improve efficacy, stability and/or reduce immunogenicity of the RNAi agent.

One aspect of the present disclosure relates to a RNAi agent comprising at least one non-natural nucleobase. In certain embodiments, the non-natural nucleobase is difluorotolyl, nitroindolyl, nitropyrrolyl, or nitroimidazolyl. In a particular embodiment, the non-natural nucleobase is difluorotolyl. In certain embodiments, only one of the two strands contains a non-natural nucleobase. In certain embodiments, both of the strands contain a non-natural nucleobase.

In one embodiment, the first two base-pairing nucleotides on the 3′ end of the sense and/or anti-sense strand are modified. In one embodiment, the first two base-pairing nucleotides on the 3′ end of the sense and/or anti-sense strand are 2′-MOE (a 2′ MOE clamp).

In one embodiment, the 3′ terminal phosphate of the sense and/or anti-sense strands is replaced by a modified internucleoside linker.

In one embodiment, at least one nucleotide of the RNAi agent is modified.

In one embodiment, said at least one modified nucleotide is selected from among 2′ alkoxyribonucleotide, 2′ alkoxyalkoxy ribonucleotide, or 2′-fluoro ribonucleotide. In another embodiment, said at least one modified nucleotide is selected from 2′-OMe, 2′-MOE and 2′-H. In various aspects, the nucleotide subunit is chemically modified at the 2′ position of the sugar. In one aspect, the 2′ chemical modification is selected from a halo, a C1-10 alkyl, a C1-10 alkoxy, a halo, and the like. In specific aspects, the 2′ chemical modification is a C1-10 alkoxy selected from —OCH₃ (i.e., “OMe”), —OCH₂CH₃ (i.e., “OEt”) or —CH₂OCH₂CH₃ (i.e., methoxyethyl or “MOE”); or is a halo selected from F.

In various embodiments, one or more nucleotides is modified or is DNA or is replaced by a peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), and/or unlocked nucleic acid (UNA); and/or at least one nucleotide comprises a modified internucleoside linker (e.g., wherein at least one phosphate of a nucleotide is replaced by a modified internucleoside linker), wherein the modified internucleoside linker is selected from phosphorothioate, phosphorodithioate, phosphoramidate, boranophosphonoate, an amide linker, and a compound of formula (I) (as described elsewhere herein).

In some embodiments, the RNAi agent to PRMT5 is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

Kits for RNAi synthesis are commercially available, e.g., from New England Biolabs and Ambion.

A suitable RNAi agent can be selected by any process known in the art or conceivable by one of ordinary skill in the art. For example, the selection criteria can include one or more of the following steps: initial analysis of the MZF1, AMH, TIMP1, or GNMT gene sequence and design of RNAi agents; this design can take into consideration sequence similarity across species (human, cynomolgus, mouse, etc.) and dissimilarity to other (non-MZF1, AMH, TIMP1, or GNMT) genes; screening of RNAi agents in vitro (e.g., at 10 nM in cells); determination of EC50 in Hela cells; determination of viability of various cells treated with RNAi agents, wherein it is desired that the RNAi agent to MZF1, AMH, TIMP1, or GNMT not inhibit the viability of these cells; testing with human PBMC (peripheral blood mononuclear cells), e.g., to test levels of TNF-alpha to estimate immunogenicity, wherein immunostimulatory sequences are less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein Immunostimulatory sequences are less desired; determination of gene knockdown in vivo using subcutaneous tumors in test animals; MZF1, AMH, TIMP1, or GNMT target gene modulation analysis, e.g., using a pharmacodynamic (PD) marker, and optimization of specific modifications of the RNAi agents.

### Pharmaceutical Composition

Also disclosed is a pharmaceutical composition comprising a disclosed MZF1, AMH, TIMP1, or GNMT inhibitor in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutically acceptable carriers include any and all suitable solvents, dispersion media, coatings, antibacterial and antifungal agents, isotonicity agents, antioxidants and absorption delaying agents, and the like that are physiologically compatible with a bispecific antibody of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

Pharmaceutical compositions may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole, butylated hydroxytoluene, lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical antibodies (e.g. nanobodies) may also comprise isotonicity agents, such as sugars, polyalcohols, such as mannitol, sorbitol, glycerol or sodium chloride in the compositions.

The pharmaceutical compositions may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The bispecific antibodies may be prepared with carriers that will protect the bispecific antibody against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art.

Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

### Methods of Treatment

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for the disease. Thus, the method can further comprise identifying a subject at risk for the disease prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, a therapeutic antibody (e.g. nanobody) is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule containing lenalidomide administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

The disclosed compositions may also be administered in combination therapy, i.e., combined with other therapeutic agents relevant for the disease or condition to be treated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: DNA Methylation and Protein Signatures Associate with Clonal Hematopoiesis Expansion Rate

### Introduction

Clonal hematopoiesis of indeterminate potential (CHIP) is an age-related phenomenon characterized by somatic mutations in hematopoietic stem cells that confer a selective advantage and cause the formation of a distinct sub-clonal cell population (Heuser, M., et al. Dtsch. Ärztebl. Int. 2016 113:317-322). This phenomenon increases in prevalence over the age of 40 and occurs in ˜10% of those above the age of 70. CHIP confers an increased risk of hematological cancer, cardiovascular disease (Argüelles, O. C., et al. J. Am. Coll. Cardiol. 2020 75:671-671), chronic obstructive pulmonary disorder (Miller, P. G., et al. Blood 2022 139:357), an increase in overall mortality (Genovese, G., et al. N. Engl. J. Med. 2014 371:2477-2487; Jaiswal, S., et al. N. Engl. J. Med. 2014 371:2488-2498), and is more prevalent in individuals infected with HIV (Bick, A. G., et al. Sci. Rep. 2022 12:577). Mutations driving CHIP are found most frequently in the DNMT3A, TET2, and ASXL1 genes (Heuser, M., et al. Dtsch. Ärztebl. Int. 2016 113:317-322).

CHIP sub-clones are defined by a minimum variant allele frequency (VAF) of 2%, but increased VAF>10% is associated with greater risk for hematologic cancer and cardiovascular disease (Jaiswal, S., et al. N. Engl. J. Med. 2014 371:2488-2498; Jaiswal, S., et al. N. Engl. J. Med. 2017 377:111-121). In addition, although CHIP is defined by a VAF of at least 2%, clones with a VAF below 2% are present in around 95% of individuals over the age of 50 (Young, A. L., et al. Nat. Commun. 2016 7:12484; Bowman, R. L., et al. Cell Stem Cell 2018 22:157-170). Identifying which individuals among this larger group have clones that grow at a faster rate, and therefore are more likely to progress to large clones and adverse outcomes, is crucial to advancing clinical care of CHIP. Driver mutation (Uddin, M. M., et al. Immun. Ageing 2022 19:23; Watson, C. J., et al. Science 2020 367:1449-1454), cancer therapies (Bolton, K. L., et al. Nat. Genet. 2020 52:1219-1226), and metabolic syndrome (van Deuren, R. C. et al. bioRxiv 2021.05.12.443095) likely impact CHIP expansion; however, additional studies are needed to evaluate determinants of clonal expansion rate in the population at large. CHIP is associated with aging and inflammation, but it is not yet understood if these factors affect CHIP clonal expansion rate (Park, S. J., et al. Curr. Stem Cell Rep. 2018 4:209-219; Nachun, D., et al. Aging Cell 2021 20: e13366; Fuster, J. J., et al. Science 2017 355:842-847; Fidler, T. P., et al. Nature 2021 592:296-301). The hypothesis that aging and inflammation lead to faster clonal expansion rates in patients with CHIP was tested using human genomics.

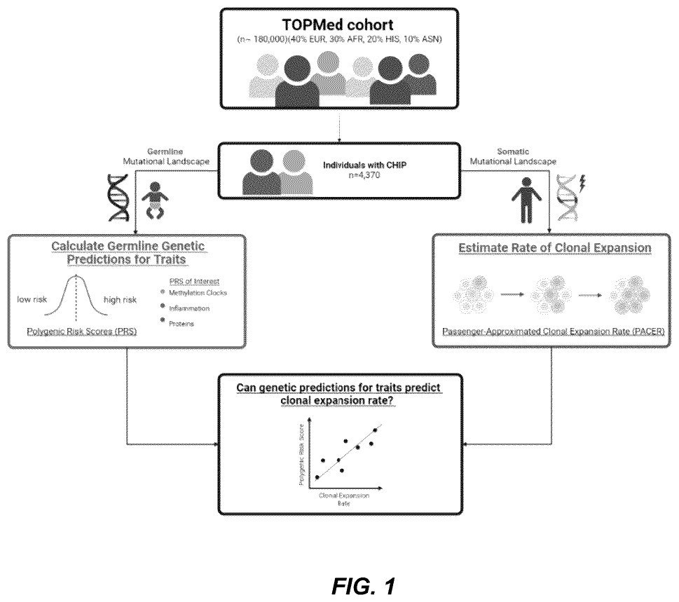

To test this hypothesis, the Passenger-Approximated Clonal Expansion Rate (PACER) method was leveraged to estimate CHIP expansion rate in 4,370 individuals from the NHLBI TOPMed cohort (Weinstock, J. S., et al. bioRxiv 2021.12.10.471810). PACER uses the steady accumulation of passenger mutations throughout the lifespan to benchmark the onset of clonal expansion; clones carrying more passenger mutations began to expand later in life (Osorio, F. G., et al. Cell Rep. 2018 25:2308-2316.e4; Mitchell, E. et al. bioRxiv 2021.08.16.456475; Williams, N. et al. bioRxiv 2020.11.09.374710). Using germline-predicted traits, the effects of DNA methylation age was measured and inflammation on clonal expansion was estimated by PACER. An unbiased proteome wide search was then carried out to identify genetically predicted circulating proteins associated with clonal expansion rate (FIG. 1). Collectively, these analyses expand our understanding of CHIP clonal expansion in humans.

### Results

### Cohort Characteristics:

As previously reported (Weinstock, J. S., et al. bioRxiv 2021.12.10.471810), 4,370 individuals with a single CHIP mutation and adequate covariate data in the TOPMed cohort were identified. 61% were female, and the average age of the cohort was 68 years. The most common driver genes were DNMT3A (46%), TET2 (15%), ASXLI (7%), and PPM1D (4%), and average variant allele frequency (VAF) was 18.7%.

### DNA Methylation Age

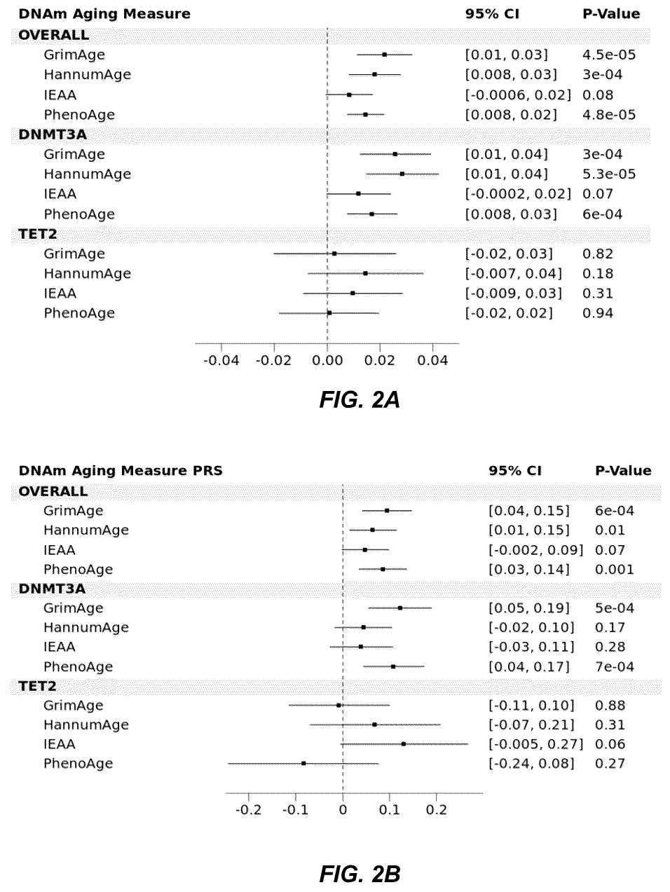

DNA methylation biomarkers of aging were significantly associated with CHIP clonal expansion. To quantify DNA methylation aging, epigenetic clocks were calculated (PhenoAge, GrimAge, and HannumAge), which are an established measure of biological age and use hyper/hypomethylation at CpG sites to quantify epigenetic alteration of aging in an individual, and an epigenetic clock derivative, Intrinsic Epigenetic Age Acceleration (IEAA) on a subset of the cohort (n=297) (Field, A. E. et al. Mol. Cell 2018 71:882-895). DNA methylation age was significantly associated with PACER for overall CHIP in PhenoAge (p=4.8×10⁻⁵), HannumAge (p=3.0×10⁻⁴), and GrimAge (p=4.5×10⁻⁵), but not in IEAA (p=0.08). Gene-specific analyses of DNMT3A and TET2 CHIP were all directionally consistent with the overall data (FIG. 2A). Two additional DNA methylation-based markers of epigenetic aging, granulocyte proportions and PAI-1, were not associated with PACER (Table 1).

TABLE 1
Regressions of clonal expansion rate with
additional measures of epigenetic aging.

| Epigenetic Aging Measure | CHIP Gene | Beta Value | P-Value |
|---|---|---|---|
| Granulocyte Proportion | Overall | −8.03 | 0.75 |
| | DNMT3A | 29.67 | 0.30 |
| | TET2 | −45.55 | 0.48 |
| PAI-1 | Overall | 14.84 | 0.22 |
| | DNMT3A | 21.25 | 0.10 |
| | TET2 | 25.94 | 0.47 |
| PAI-1 PRS | Overall | 1.46 | 0.61 |
| | DNMT3A | 5.33 | 0.11 |
| | TET2 | 1.36 | 0.83 |

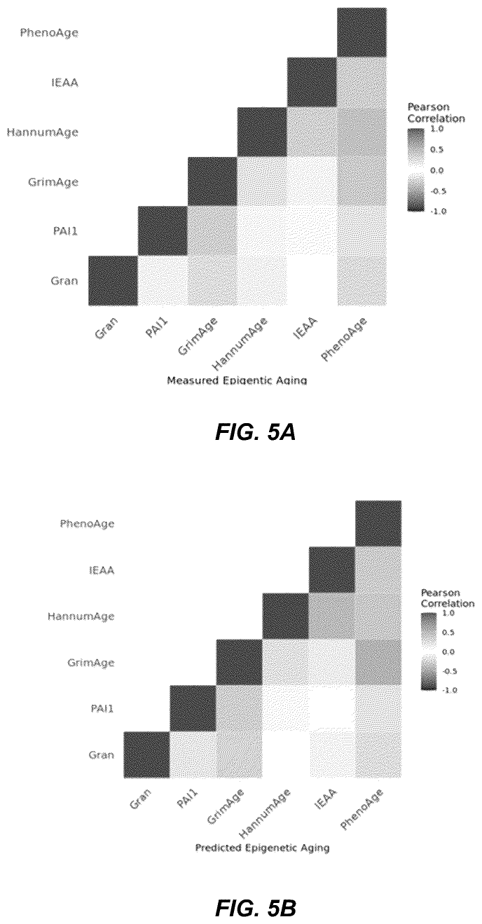

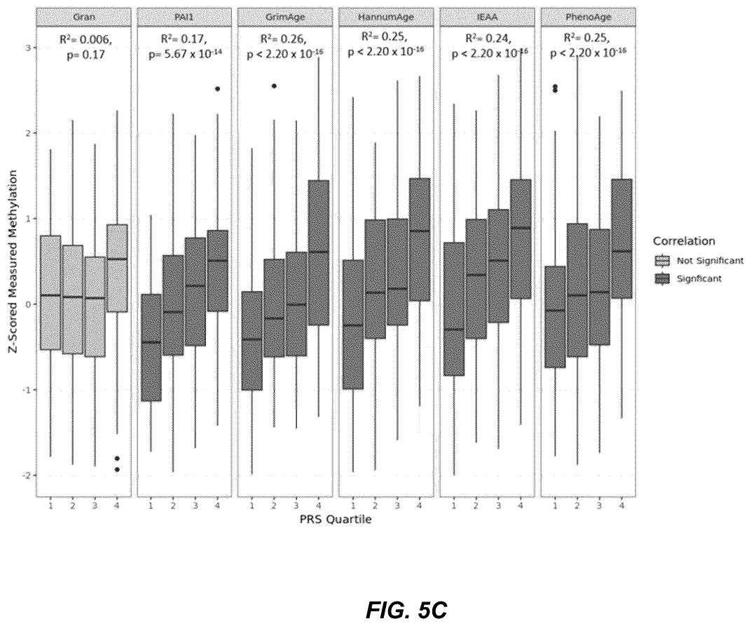

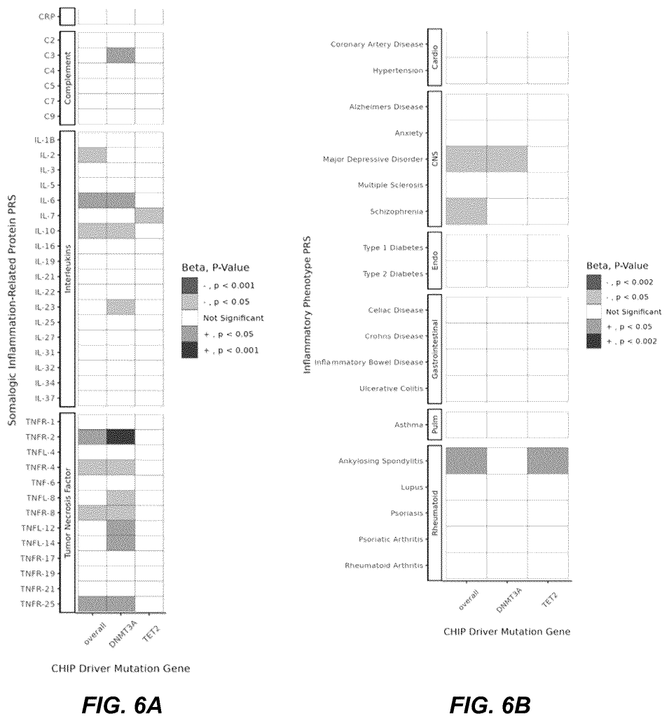

The GWAS summary statistics was then used for the six DNA methylation biomarkers of aging in a multi-ethnic cohort to calculate polygenic risk scores (PRS) for the entire cohort (n=4,370) (McCartney, D. L. et al. Genome Biol. 2021 22:194). PRS's were predictive of measured epigenetic age in our cohort in each of the six tested methylation-based markers except granulocyte proportions (FIG. 5). Predicted DNA methylation aging was significantly associated with PACER clonal expansion rate for overall CHIP in PhenoAge (p=1.2×10⁻³), HannumAge (p=0.01), and GrimAge (p=6.0×10⁻⁴), but not in IEAA (p=0.07). In gene-specific analyses, DNMT3A CHIP clonal expansion rate was significantly associated with predicted PhenoAge (7.0×10⁻⁴) and GrimAge (5.0×10⁻⁴), but TET2-specific CHIP did not have any significant associations. (FIG. 2B).

### Inflammation

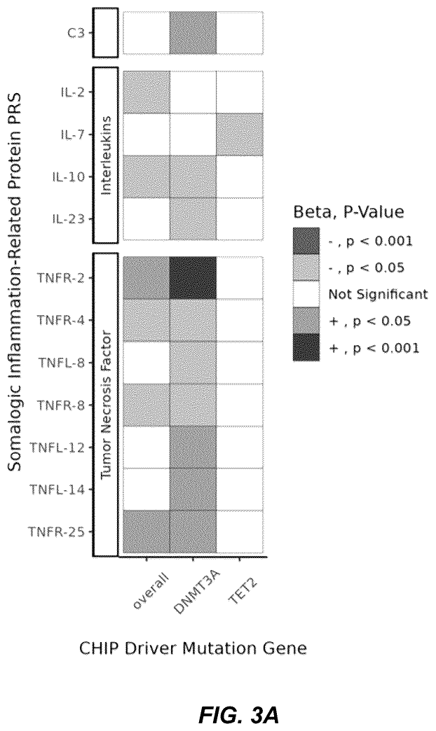

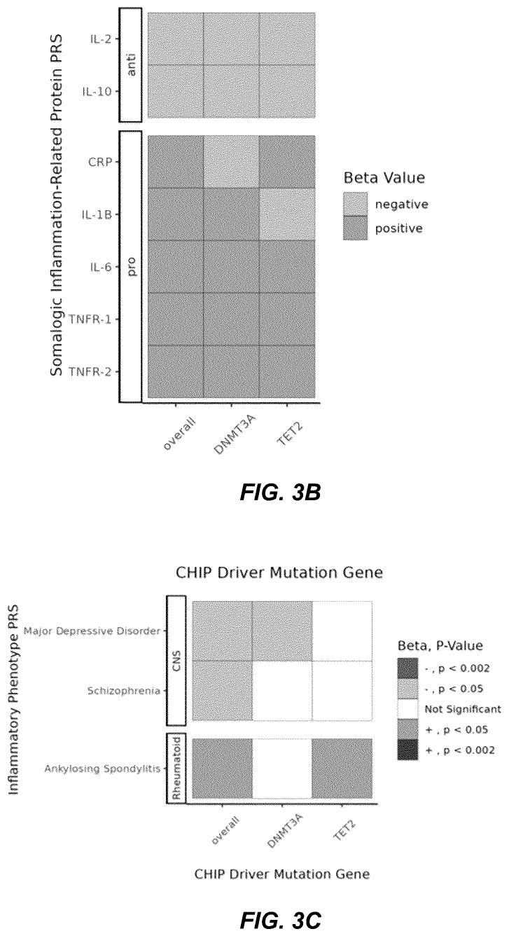

Associations between germline genetic prediction of inflammation-related traits and clonal expansion rate was tested. We leveraged PRS's derived from recent large-scale GWAS studies to predict inflammatory lab values, disease phenotypes, and circulating inflammatory cytokines (Table 2). There were no significant associations between any of the genetically predicted inflammatory lab values, diseases or cytokines and PACER and overall CHIP after correction for multiple hypothesis testing. Only one significant hit was identified for DNMT3A-CHIP in TNFR-2 in the gene-specific analyses (FIG. 3A-3C). There was a pattern where proteins with an established pro-inflammatory effect had positive associations with expansion rate, and proteins with anti-inflammatory effects had negative associations. This association was significant by sign test (p=0.015) (FIG. 3B).

### Circulating Proteins

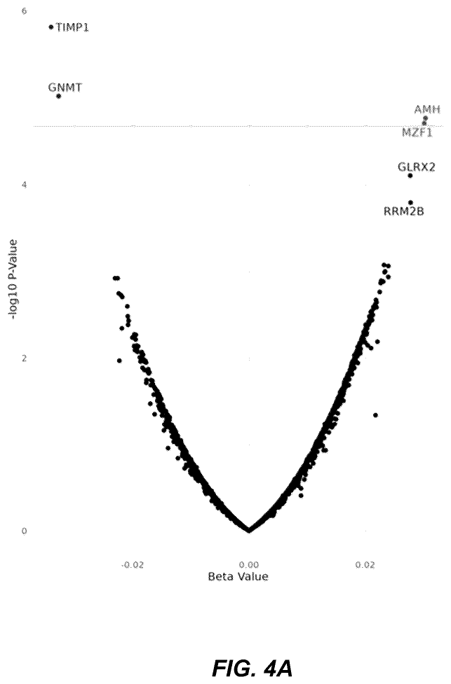

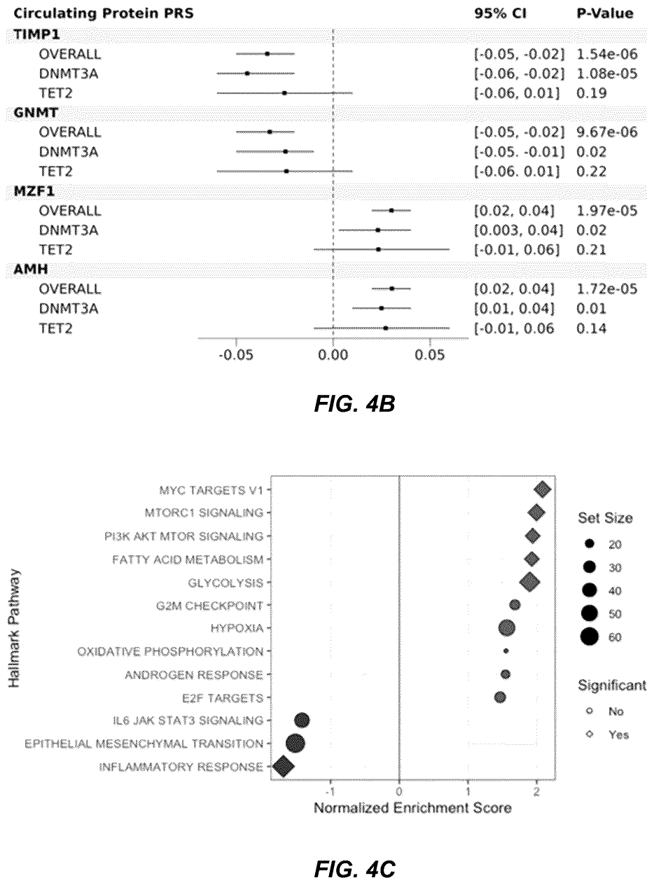

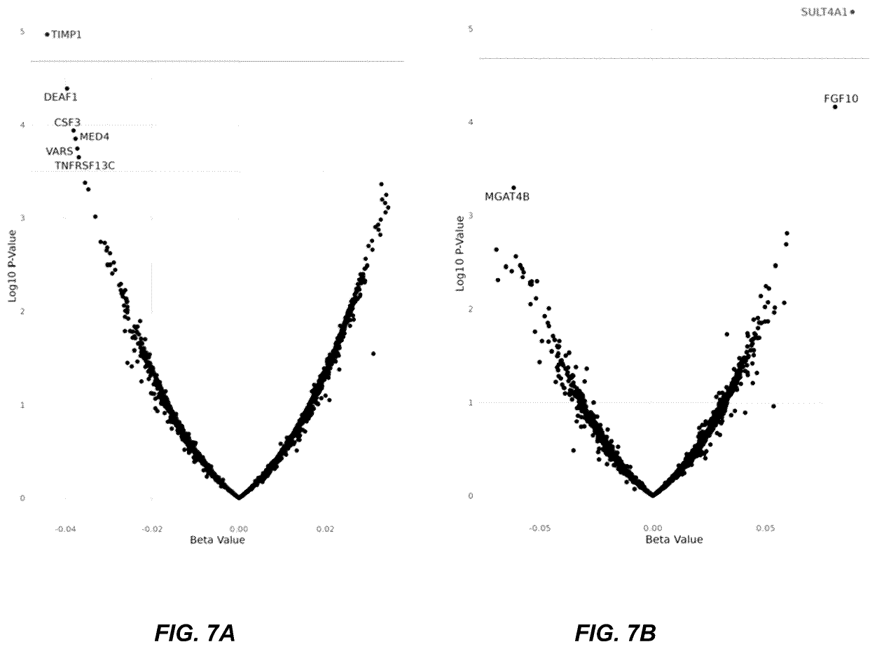

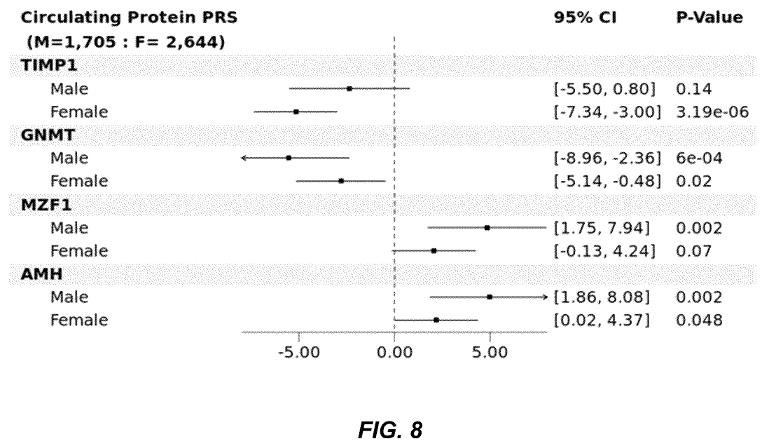

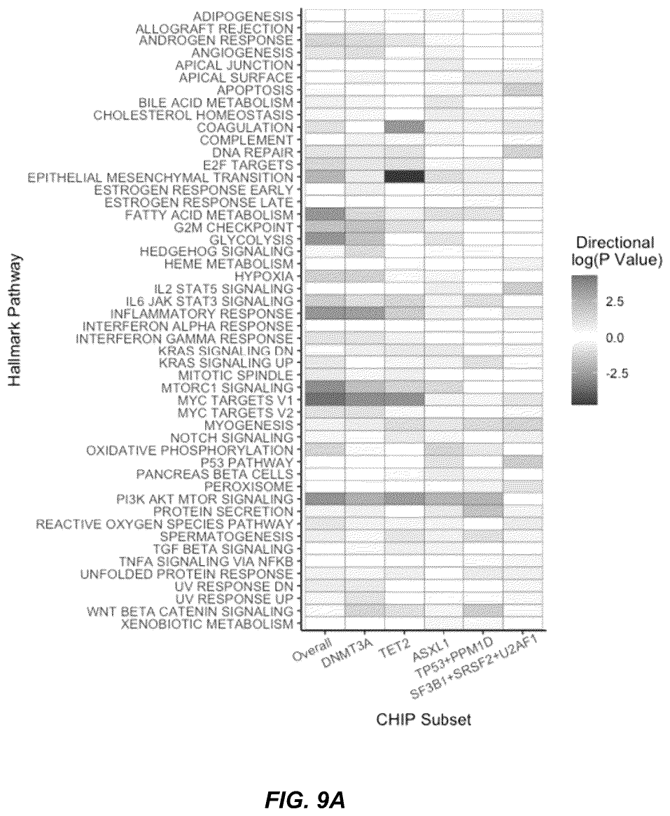

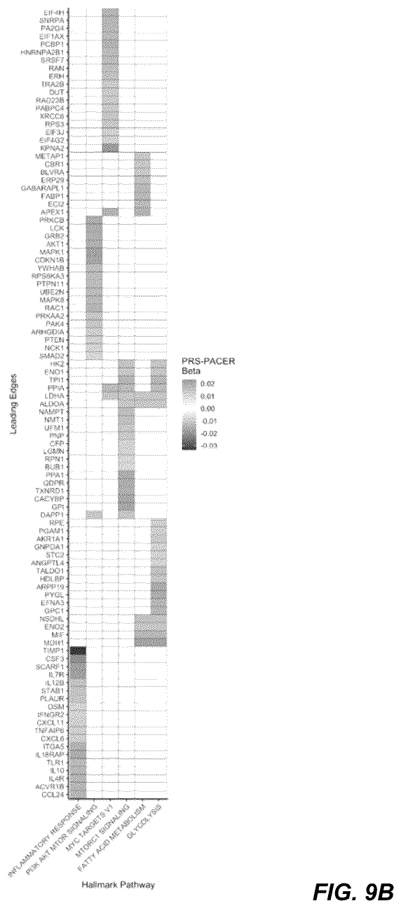

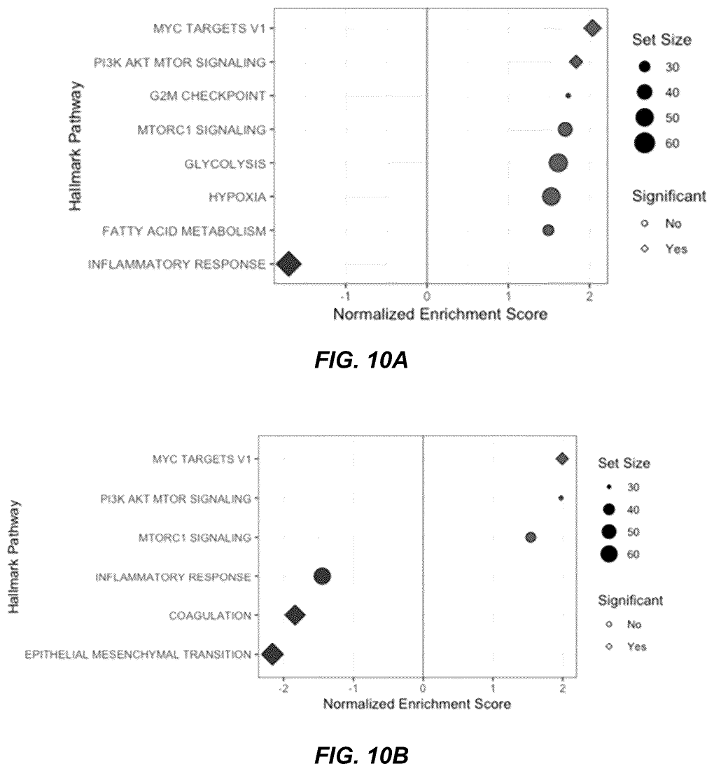

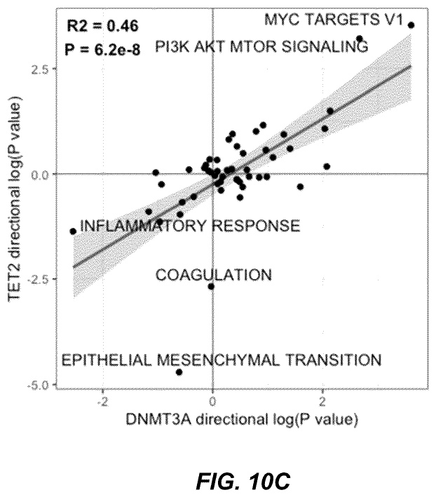

an unbiased search across the circulating proteome was then performed. We computed genetic predisposition to 2,384 circulating proteins quantified on the Somalogic platform and tested their association with PACER (FIG. 4, Table 3). Four proteins reached the Bonferroni-adjusted statistical significance threshold (p<=2.09×10⁻⁵) in an analysis of overall CHIP. Genetically predicted values for TIMP metallopeptidase inhibitor 1 (TIMP1) (p=1.5×10⁻⁶) and glycine N-methyltransferase (GNMT) (p=9.67×10⁻⁶) were negatively associated with PACER, while genetically predicted values for Anti-Mullerian Hormone (AMH) (p=1.72×10⁻⁵) and Myeloid Zinc Finger 1 (MZF1) (p=1.97×10⁻⁵) were significantly positively associated with PACER (FIG. 4A). Associations were directionally consistent in driver gene-specific analyses (FIGS. 4B, 7). Directionality was also consistent in a sex-stratified regression analysis, although the effect size was larger in females for TIMP1 and in males for GNMT, MZF1, and AMH (FIG. 8). To further contextualize these findings, gene set enrichment analysis (GSEA) was performed on the circulating protein dataset (FIGS. 4C, 9). Metabolic and mTOR signaling pathways were significantly enriched in our dataset, while the inflammatory response program was significantly negatively associated with PACER expansion. Leading edge analysis identifies common drivers of these GSEA results (FIG. 9). In driver gene-specific analyses of PACER determinants, DNMT3A and TET2 CHIP had correlated results largely mimicking those of the larger analysis (FIG. 10). TET2 CHIP was uniquely negatively associated with coagulation and epithelial-mesenchymal transition.

TABLE 2
Description of inflammation PRS derivation.

| Inflammatory | | | | Genetic | # of | Method of |
|---|---|---|---|---|---|---|
| Phenotype | Year | Cases | Controls | Ancestry | SNPs | PRS Calculation |
| Coronary | 2020 | 91.316 | 300,925 | European, | 5,167,567 | PLINK |
| Artery Disease | | | | Japanese | | |
| Hypertension | 2017 | 76,501 | 64,384 | European | 107 | PLINK |
| Alzheimer's | 2020 | 71,880 | 383,378 | European | 13,367,301 | PRScs & |
| Disease | | | | | | PLINK |
| Anxiety | 2016 | 7,016 | 14,745 | European | 6,500,000 | PRScs & |
| | | | | | | PLINK |
| Major | 2019 | 246,363 | 561,190 | European | 10,000 | PRScs & |
| Depressive | | | | | | PLINK |
| Disorder | | | | | | |
| Multiple | 2021 | 142 | 12,826 | European | 127 | PLINK |
| Sclerosis | | | | | | |
| Schizophrenia | 2018 | 40,675 | 64,643 | European | 8,000,000 | PRScs & |
| | | | | | | PLINK |
| Type 1 | 2021 | 22,153 | 37,374 | European, | 715,631 | PLINK |
| Diabetes | | | | African, | | |
| | | | | Admixed | | |
| Type 2 | 2020 | 9,978 | 13,348 | European | 8,919,079 | PRScs & |
| Diabetes | | | | | | PLINK |
| Celiac Disease | 2011 | 12,041 | 12,228 | European | 40 | PLINK |
| Crohn's | 2017 | 22,575 | 46,693 | European, | 36 | PLINK |
| Disease | | | | East Asian, | | |
| | | | | Indian, | | |
| | | | | Iranian | | |
| Inflammatory | 2015 | 42,950 | 53,536 | European, | 167 | PRScs & |
| Bowel Disease | | | | East Asian, | | PLINK |
| | | | | Indian, | | |
| | | | | Iranian | | |
| Ulcerative | 2015 | 20,417 | 52,230 | European, | 29 | PLINK |
| Colitis | | | | East Asian, | | |
| | | | | Indian, | | |
| | | | | Iranian | | |
| Asthma | 2020 | 88,486 | 9,415,011 | European, | 312 | PLINK |
| | | | | Hispanic, | | |
| | | | | African | | |
| | | | | American, | | |
| | | | | East Asian | | |
| Ankylosing | 2013 | 10,619 | 15,145 | European, | 128,935 | PRScs & |
| Spondylitis | | | | East Asian | | PLINK |
| Lupus | 2015 | 7,219 | 15,991 | European | 644,674 | PRScs & |
| | | | | | | PLINK |
| Psoriasis | 2017 | 6,463 | 6,096 | European | 63 | PRScs & |
| | | | | | | PLINK |
| Psoriatic | 2018 | 835 | 1,558 | Spanish, | 38 | PLINK |
| Arthritis | | | | North | | |
| | | | | American | | |
| Rheumatoid | 2014 | 29,880 | 73,758 | European, | 101 | PRScs & |
| Arthritis | | | | East Asian | | PLINK |

TABLE 3
Results for top 50 proteins in Somalogic associations.

| | | Beta | | INTERVAL |
|---|---|---|---|---|
| Protein | Gene | Value | P-Value | R2 |
| Metalloproteinase inhibitor 1 | TIMP1 | −0.03 | 1.54 × 10−6 | 0.02 |
| Glycine N-methyltransferase | GNMT | −0.03 | 9.67 × 10−6 | 0.07 |
| Muellerian-inhibiting factor | AMH | 0.03 | 1.72 × 10−5 | 0.02 |
| Myeloid zinc finger 1 | MZF1 | 0.03 | 1.97 × 10−5 | 0.01 |
| Glutaredoxin-2 | GLRX2 | 0.03 | 7.90 × 10−5 | 0.10 |
| Ribonucleoside-diphosphate | RRM2B | 0.03 | 1.62 × 10−4 | 0.07 |
| reductase subunit M2 B | | | | |
| Importin subunit alpha-1 | KPNA2 | 0.02 | 8.56 × 10−4 | 0.03 |
| Low-density lipoprotein receptor | LDLRAD4 | 0.02 | 8.80 × 10−4 | 0.10 |
| class A domain-containing | | | | |
| protein 4 | | | | |
| Ras-related protein Rab-26 | RAB26 | 0.02 | 1.02 × 10−3 | 0.07 |
| Mediator of RNA polymerase II | MED1 | 0.02 | 1.04 × 10−3 | 0.10 |
| transcription subunit 1 | | | | |
| Aldose reductase | AKR1B1 | 0.02 | 1.18 × 10−3 | 0.06 |
| Adseverin | SCIN | −0.02 | 1.21 × 10−3 | 0.04 |
| Ligand-dependent nuclear | LCORL | −0.02 | 1.22 × 10−3 | 0.02 |
| receptor corepressor-like protein | | | | |
| Adenylosuccinate synthetase | ADSS | 0.02 | 1.30 × 10−3 | 0.09 |
| isozyme | | | | |
| 5-formyltetrahydrofolate cyclo- | MTHFS | 0.02 | 1.33 × 10−3 | 0.18 |
| ligase | | | | |
| Low-density lipoprotein receptor- | LRP1B | 0.02 | 1.38 × 10−3 | 0.17 |
| related protein 1B (11275) | | | | |
| ADP-ribosylation factor-binding | GGA3 | 0.02 | 1.75 × 10−3 | 0.08 |
| protein GGA3 | | | | |
| Low-density lipoprotein receptor- | LRP1B | −0.02 | 1.82 × 10−3 | 0.17 |
| related protein 1B (7640) | | | | |
| Glypican-6 | GPC6 | −0.02 | 1.90 × 10−3 | 0.02 |
| Deformed epidermal | DEAF1 | −0.02 | 2.00 × 10−3 | 0.05 |
| autoregulatory factor 1 homolog | | | | |
| Alpha-(1,3)-fucosyltransferase 10 | FUT10 | 0.02 | 2.16 × 10−3 | 0.112 |
| Mitogen-activated protein kinase 1 | MAPK1 | 0.02 | 2.31 × 10−3 | 0.08 |
| Protein S100-A11 | S100A11 | 0.02 | 2.42 × 10−3 | 0.10 |
| Inositol monophosphatase 3 | IMPAD1 | −0.02 | 2.56 × 10−3 | 0.08 |
| Cyclin-dependent kinase inhibitor 1B | CDKN1B | 0.02 | 2.61 × 10−3 | 0.08 |
| Malate dehydrogenase | MDH1 | 0.02 | 2.63 × 10−3 | 0.04 |
| Tumor necrosis factor alpha- | TNFAIP8 | 0.02 | 2.71 × 10−3 | 0.14 |
| induced protein 8 | | | | |
| 40S ribosomal protein S4 | RPS4X | 0.02 | 2.89 × 10−3 | 0.16 |
| Macrophage-capping protein | CAPG | 0.02 | 2.99 × 10−3 | 0.19 |
| Calcyclin-binding protein | CACYBP | 0.02 | 3.17 × 10−3 | 0.11 |
| Pyruvate kinase PKM | PKM2 | 0.02 | 3.21 × 10−3 | 0.06 |
| Glucose-6-phosphate isomerase | GPI | 0.02 | 3.25 × 10−3 | 0.13 |
| IST1 homolog | IST1 | 0.02 | 3.30 × 10−3 | 0.07 |
| Dynactin subunit 2 | DCTN2 | −0.02 | 3.34 × 10−3 | 0.02 |
| Sialic acid-binding Ig-like lectin 14 | SIGLEC14 | 0.02 | 3.36 × 10−3 | 0.43 |
| Chromobox protein homolog 7 | CBX7 | 0.02 | 3.65 × 10−3 | 0.07 |
| Complement factor B | CFB | 0.02 | 3.67 × 10−3 | 0.13 |
| Toll/interleukin-1 receptor | TIRAP | 0.02 | 3.69 × 10−3 | 0.14 |
| domain-containing adapter | | | | |
| protein | | | | |
| WNT1-inducible-signaling | WISP3 | −0.02 | 3.73 × 10−3 | 0.03 |
| pathway protein 3 | | | | |
| Heterogeneous nuclear | HNRNPDL | 0.02 | 3.84 × 10−3 | 0.16 |
| ribonucleoprotein D-like | | | | |
| Signal-regulatory protein beta-2 | SIRPB2 | 0.02 | 3.91 × 10−3 | 0.08 |
| ADP-ribosylation factor-like | ARL3 | 0.02 | 4.12 × 10−3 | 0.11 |
| protein 3 | | | | |
| MYC Associated Factor X | MAX | 0.02 | 4.15 × 10−3 | 0.07 |
| Granulocyte colony-stimulating | CSF3 | −0.02 | 4.15 × 10−3 | 0.27 |
| factor | | | | |
| RAC-alpha serine/threonine- | AKT1 | 0.02 | 4.19 × 10−3 | 0.07 |
| protein kinase | | | | |
| ATP-dependent RNA helicase | DDX19B | 0.02 | 4.46 × 10−3 | 0.12 |
| DDX19B | | | | |
| RAC-beta serine/threonine- | AKT2 | 0.02 | 4.46 × 10−3 | 0.09 |
| protein kinase | | | | |
| von Willebrand factor A domain- | VWA1 | −0.02 | 4.56 × 10−3 | 0.04 |
| containing protein 1 | | | | |
| Maspardin | SPG21 | 0.02 | 4.57 × 10−3 | 0.13 |
| DiGeorge syndrome critical | DGCR14 | 0.020013 | 4.61 × 10−3 | 0.07 |
| region 14 | | | | |

### Discussion

CHIP is a prevalent disease entity that increases all-cause mortality, yet current understanding of the factors that contribute to clonal expansion rate is limited. We calculated the estimated clonal expansion rate of ˜4,500 individuals with CHIP from the TOPMed database using the PACER technique and measured associations with genetic risk for methylation clocks, inflammation, and circulating protein levels. We have shown that genetic predisposition to aging increases expansion rate and identified four candidate proteins that affect CHIP expansion rate.

Three epigenetic clocks, GrimAge, PhenoAge, and HannumAge, were significantly positively associated with clonal expansion rate, quantified both by measured methylation data and PRS. The GrimAge methylation clock is a time-to-death prediction metric that was developed with a two-stage approach based on a DNA methylation-based estimator of smoking pack-years and seven DNA methylation-based markers for severe plasma proteins associated with metabolic syndrome and inflammation, including TIMP metallopeptidase inhibitor 1 (TIMP1). It is predictive of time to death, time to coronary heart disease, and time to cancer (Lu, A. T. et al. Aging 2019 11:303-327). The PhenoAge methylation clock is based on biological aging processes such as pro-inflammatory pathways, DNA damage response, mitochondrial signatures, and decreased transcription/translation (Levine, M. E. et al. Aging 2018 10:573-591). The HannumAge methylation clock uses a set of 71 methylation markers which are predictive of age and reside near genes associated with age-related conditions such as Alzheimer's Disease and oxidative stress (Hannum, G. et al. Mol. Cell 2013 49:359-367). Intrinsic Epigenetic Age Acceleration (IEAA) is a derivative of the Horvath epigenetic clock that controls for white blood cell proportions and has been previously linked to CHIP status, but was not significantly associated with clonal expansion rate in this study (Robertson, N. A. et al. Curr. Biol. 2019 29: R786-R787). Further analyses into the components of these significant methylation clocks may explain individual factors that influence clonal expansion rate. Overall, these observations are concordant with murine models suggesting clonal expansion accelerates in aged bone marrow microenvironment (SanMiguel, J. M. et al. Cancer Discov. 2022 12:2763-2773).

Surprisingly, no individual inflammatory proteins or phenotypes were significantly associated with clonal expansion rate in overall CHIP. Inflammation and CHIP appear to be linked based on previous studies; CHIP enacts disease at least in part through inflammation (Marnell, C. S. et al. J. Mol. Cell. Cardiol. 2021 161:98-105; Bick, A. G. et al. Circulation 2020 141:124-131). Additionally, an inflammatory environment can increase likelihood of CHIP emergence, which is thought to be driven by “inflammaging”, low grade chronic inflammation associated with aging (Kristinsson, S. Y. et al. J. Clin. Oncol. 2011 29:2897-2903; Cho, R. H. et al. Blood 2008 111:5553-5561; Franceschi, C. et al. Ann. N. Y. Acad. Sci. 2000 908:244-254; Cook, E. K. et al. Exp. Hematol. 2020 83:85-94). However, in our study, no significant associations between germline inflammation risk and clonal expansion rate were found. However, inflammatory proteins that have an established pro- or anti-inflammatory effect (Dinarello, C. A. et al. Chest 2000 118:503-508; Zhang, J.-M. et al. Int. Anesthesiol. Clin. 2007 45:27-37) were directionally consistent. IL-6, IL-1B, TNFR-1, TNFR-2, and CRP showed a positive effect on clonal expansion rate, and IL-2 and IL-10 showed a negative effect. However, most GWASs for these inflammatory markers are small and underpowered and identified GWS loci explain very little of estimated heritability. It is possible that a study with increased power could better identify risk modulators in this area . . .

Our data also raises the possibility that inflammatory stimuli associated with clonal expansion may be CHIP driver gene specific. A recent study characterizing a mouse model of DNMT3A CHIP, found that TNFα-induced signaling in the aged bone marrow promotes DNMT3A CHIP clonal expansion (SanMiguel, J. M. et al. Cancer Discov. 2022 12:2763-2773). Although there was no association of TNFα with clonal expansion in CHIP overall, there was evidence for clonal expansion in DNMT3A CHIP, consistent with the murine data.

Similarly, chronic inflammatory disease risk was also not significantly associated with clonal expansion rate, which could be due to the multifactorial contributions to these conditions. While germline risk of inflammation was used in the analysis, any relationship between CHIP and inflammation may be primarily environmental or acquired. Nonetheless, these results do not indicate a significant association between baseline inflammation and CHIP expansion rate.

Four circulating protein levels associated with clonal expansion rate. TIMP1 and GNMT were significantly associated with decreased clonal expansion rate, while AMH and MZF1 were significantly associated with increased clonal expansion rate. TIMP1 is a matrix metalloproteinase inhibitor known to promote cell proliferation and have anti-apoptotic functions. Elevated TIMP1 levels are associated with poorer outcomes and faster progression in colorectal cancer (Gong, Y. et al. PLOS ONE 2013 8: e77366; Song, G. et al. J. Exp. Clin. Cancer Res. 2016 35:148), pancreatic cancer (Schoeps, B. et al. Cancer Res. 2021 81:3568-3579), and small cell lung cancer (Pesta, M. et al. Anticancer Res. 2011 31:4031-4038). However, the effect is in the opposite direction in our findings. GNMT is involved in methionine breakdown and processing of toxic compounds in the liver. GNMT is associated with worse outcomes in prostate cancer (Song, Y. H. et al. Mod. Pathol. 2011 24:1272-1280) but also acts as a tumor suppressor across multiple cancers (DebRoy, S. et al. PLOS ONE 2013 8: e70062), which is consistent with the direction here. AMH is involved in sex differentiation and reproductive system homeostasis. Higher levels of AMH have been implicated in ovarian cancer (La Marca, A. et al. Hum. Reprod. Update 2007 13:265-273), but alternative large-scale studies have failed to find this association (Jung, S. et al. Int. J. Cancer 2018 142:262-270). The role of AMH in clonal expansion rate may help explain the difference in CHIP prevalence between sexes. MZF1 is a transcription regulator involved in hematopoietic development. Higher MZF1 levels are linked to worse cancer prognosis (Brix, D. M. et al. Cells 2020 9:223), but have also been shown to decrease viability and proliferation of lymphoma cells (Vishwamitra, D. et al. Mol. Cancer 2015 14:53) and inhibit hematopoietic development along the myeloid lineage (Perrotti, D. et al. Mol. Cell. Biol. 1995 15:6075-6087). These identified genetically predicted circulating proteins may serve as candidates for further mechanistic study of CHIP expansion.

As discovery power might be limited for individual circulating proteins, and the variance explained by the protein PRSs is low, GSEA was leveraged to identify pathways associated with clonal expansion. Gene sets representative of increased metabolic activity associated with faster CHIP expansion rate which is concordant with prior observational data suggesting that metabolic syndrome accelerates clonal hematopoiesis (van Deuren, R. C. et al. bioRxiv 2021.05.12.443095). Conversely, the inflammation gene set was associated with slower CHIP expansion. This result is in opposition to our curated inflammatory set (FIG. 3), driven by leading edge genes with generally less well-defined inflammatory functions. These results highlight that inflammation may be playing a role in both CHIP expansion and clonal selection perhaps through immune surveillance (Cook, E. K. et al. Exp. Hematol. 2020 83:85-94; Avagyan, S. et al. Science 2021 374:768-772; Swann, J. B. et al. J. Clin. Invest. 2007 117:1137-1146).

### CONCLUSIONS

In summary, CHIP is a prevalent disease process that increases overall mortality in those with larger clones, but little is known about what factors increase the rate of clonal expansion. Here, we identify determinants of clonal expansion rate. These results suggest that biological aging, metabolism, inflammation, and four unique circulating proteins affect CHIP expansion rate. These identified factors have the potential to act as therapeutic targets to modulate CHIP clonal expansion and improve outcomes for patients with CHIP.

### Methods

### Study Cohort

As part of 51 studies that contributed to the Freeze 8 TOPMed program, 127,946 individual samples underwent whole genome sequencing (WGS) as previously described (Taliun, D., et al. Nature 2021 590:290-299). All included individuals provided informed consent to participate. For the 82,807 samples with age data available, mean age was 52.5, and the samples come from diverse ancestral backgrounds, 40% European, 32% African, 16% Hispanic, 10% Asian.

### CHIP Calling

From these individuals, 4,370 were identified to have CHIP through previously described methods that utilized the Mutect2 pipeline (Bick, A. G. et al. bioRxiv 782748) 0. Individuals were classified as having CHIP if there were at least three reads of a CHIP driver mutation from a list of identified CHIP mutations as previously described (Weinstock, J. S., et al. bioRxiv 2021.12.10.471810).

### PACER Score Calculation

The Passenger-Approximated Clonal Expansion Rate (PACER) method was used to approximate clonal expansion rate for each individual as previously described (Weinstock, J. S., et al. bioRxiv 2021.12.10.471810). Passenger mutation count is used as a proxy for the passage of time to approximate the time of origin of the driver mutation. Relative expansion rate is estimated by regressing passenger mutation count on covariates: age at blood draw, sex, smoking status (ever/never), TOPMed study, and TCL1A genotype, which was previously implicated in clonal expansion rate (Weinstock, J. S., et al. bioRxiv 2021.12.10.471810), and the first ten principal components, a control for population stratification.

### Polygenic Risk Scoring

Polygenic risk scores (PRS) are a well-established metric to quantitatively measure an individual's risk for a phenotype due to the additive effects of common polymorphisms (Wray, N. R. et al. Genome Res. 2007 17:1520-1528). Summary statistics for the four DNA methylation k analyses were gathered from a recent multi-ethic GWAS on DNA methylation aging (McCartney, D. L. et al. Genome Biol. 2021 22:194). The trans-ethnic meta analysis that produced these summary statistics was originally performed using 36 datasets from 30 cohorts and was comprised of 40,905 individuals (34,710 European, 6,195 African). SNPs with a p-value of greater than 0.05 were removed from the score calculation for simplicity. Scores were calculated using PRScsx (Ruan, Y. et al. Nat. Genet. 2022 54:573-580) (https://github.com/getian107/PRScsx), a tool for cross-ethnic polygenic score prediction, with both the European and African reference panels used, and PLINK (Purcell, S. et al. Am. J. Hum. Genet. 2007 81:559-575) (http://pngu.mgh.harvard.edu/purcell/plink/). Scores for the proteome-wide protein panel (n=2,384 proteins) and the inflammation protein panel (n=38 proteins) were calculated using PLINK based on models from the OMICSPRED database (Xu, Y., et al. bioRxiv 2022.04.17.488593). These models were created using circulating proteins from the Somalogic panel (https://somalogic.com/) that had more than one significant GWAS hit, were trained in the INTERVAL cohort (European, n=3,175), and externally validated in the FENLAND (European (n=8,832)), MEC (Chinese (n=645), Indian (N=564), Malay (N=563)), and JHS (African American (n=1,852)) cohorts. Genetic scores were constructed using both cis and trans eQTLs. The data was downloaded in August 2021. Scores for the inflammatory diseases were calculated from large scale GWAS data. The best method for score calculation was chosen based on the number of SNPs reported in the summary statistics of each GWAS. PLINK was used to generate polygenic risk scores for chronic inflammatory diseases (Furman, D., et al. Nat. Med. 2019 25:1822-1832), Celiac Disease (Trynka, G., et al. Nat. Genet. 2011 43:1193-1201), Psoriatic Arthritis (Aterido, A., et al. Ann. Rheum. Dis. 2019 78:355-364), Ulcerative Colitis (Liu, J. Z., et al. Nat. Genet. 2015 47:979-986), Crohn's Disease (Liu, J. Z., et al. Nat. Genet. 2015 47:979-986), Type 1 Diabetes (Robertson, C. C., et al. Nat. Genet. 2021 53:962-971), Multiple Sclerosis (Barnes, C. L. K., et al. Eur. J. Hum. Genet. EJHG 2021 29:1701-1709), Hypertension (Warren, H. R., et al. Nat. Genet. 2017 49:403-415), Asthma (Han, Y., et al. Nat. Commun. 2020 11:1776), and Coronary Artery Disease (Matsunaga, H., et al. Circ Genom Precis Med. 2020 13 (3): e002670). PRScs (Ge, T., et al. Nat. Commun. 2019 10:1776) and PLINK were used to generate polygenic risk scores for Inflammatory Bowel Disease (Liu, J. Z., et al. Nat. Genet. 2015 47:979-986), Rheumatoid Arthritis (Okada, Y., et al. Nature 2014 506:376-381), Lupus (Bentham, J., et al. Nat. Genet. 2015 47:1457-1464), Ankylosing Spondylitis (Cortes, A., et al. Nat. Genet. 2013 45:730-738), Type 2 Diabetes (Cai, L., et al. Sci. Data 2020 7:393), Schizophrenia (Pardiñas, A. F., et al. Nat. Genet. 2018 50:381-389), Major Depressive Disorder (Howard, D. M., et al. Nat. Neurosci. 2019 22:343-352), Anxiety (Otowa, T., et al. Mol. Psychiatry 2016 21:1391-1399), Alzheimer's Disease (Jansen, I. E., et al. Nat. Genet. 2019 51:404-413), and Psoriasis (Tsoi, L. C., et al. Nat. Commun. 2017 8:15382) (Table 2). All polygenic risk scores were z-score scaled for further analysis.

### Gene Set Enrichment Analysis

Results of Somalogic panel PRS's association with PACER were input into GSEA using their associated gene and the Hallmark Gene Set Collection (Liberzon, A., et al. Cell Syst. 2015 1:417-425). For proteins with multiple PRS's, the mean PRS association with PACER was used for GSEA. Analyses were performed using the fgsea and msigdbr packages.

### Linear Regressions

In all regression models, age at blood draw, sex, smoking status, study, and TCL1A genotype, which was previously implicated in clonal expansion rate (Weinstock, J. S., et al. bioRxiv 2021.12.10.471810), and the first ten principal components, a control for population stratification, were included as covariates. In all models, PACER score was the outcome variable. Each of the regressions was performed within overall CHIP, DNMT3A CHIP, and TET2 CHIP. In the methylation clock analysis, linear regressions were performed for each of the four methylation clocks and two additional measures of epigenetic aging. To account for multiple testing, we used a Bonferroni corrected p-value of 0.01. In the inflammation proteins analysis, a total of 38 proteins were tested, and a corrected p-value of 0.001 was used. In the inflammatory diseases analysis, a total of 19 phenotypes were tested and a p-value of 0.002 was used. In the proteome-wide analysis, a total of 2,384 proteins were tested and a p-value of 4.2×10⁻⁴ was used. Each PRS was tested to determine if it was significantly associated with PACER score, which represents clonal expansion rate.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a subject with an increased risk of developing a cardiometabolic disease or a hematological cancer, comprising the steps of:
(a) assaying a blood, serum, or plasma sample from the subject for circulating levels of myeloid zinc finger 1 (MZF1), anti-müllerian hormone (AMH), TIMP metallopeptidase inhibitor 1 (TIMP1), glycine N-methyltransferase (GNMT), or a combination thereof; and
(b) comparing the circulating levels of MZF1, AMH, TIMP1, GNMT, or a combination thereof, to control values,
(c) detecting the presence of elevated circulating levels of MZF1, AMH, TIMP1, GNMT, or a combination thereof indicating an increased risk of developing a cardiometabolic disease and/or a hematological cancer, and
(d) administering to the subject an inhibitor of MZF1, AMH, TIMP1, GNMT, or a combination thereof.

2. The method according to claim 1, wherein the cardiometabolic disease is atherosclerosis, coronary heart disease (CHD) or ischemic stroke (IS).

3. The method according to claim 1, wherein the hematological cancer is a leukemia, a lymphoma, a myeloma or a blood syndrome.

4. The method according to claim 3, wherein the leukemia is acute myeloid leukemia (AML) or chronic myelogenous leukemia (CML).

5. The method according to claim 3, wherein the blood syndrome is myelodysplastic syndrome (MDS).

6. The method according to claim 1, wherein the subjects exhibits one or more risk factors of being a smoker, having a high level of total cholesterol or having high level of high-density lipoprotein (HDL).

7. The method of claim 1, wherein the inhibitor of MZF1, AMH, TIMP1, GNMT, or a combination thereof is an antibody.

8. The method of claim 1, wherein the inhibitor of MZF1, AMH, TIMP1, GNMT, or a combination thereof is an small molecule.

9. The method of claim 1, wherein the inhibitor of MZF1, AMH, TIMP1, GNMT, or a combination thereof is polynucleotide encoding a gRNA and Cas gene.

10. A method for treating a cardiometabolic disease or a hematological cancer, comprising administering to the subject an inhibitor of MZF1, AMH, TIMP1, GNMT, or a combination thereof.

11. The method of claim 10, wherein the inhibitor of MZF1, AMH, TIMP1, GNMT, or a combination thereof is an antibody.

12. The method of claim 10, wherein the inhibitor of MZF1, AMH, TIMP1, GNMT, or a combination thereof is an small molecule.

13. The method of claim 10, wherein the inhibitor of MZF1, AMH, TIMP1, GNMT, or a combination thereof is polynucleotide encoding a gRNA and Cas gene.

14. The method of claim 13, wherein the inhibitor of MZF1 is polynucleotide encoding a gRNA and Cas gene, wherein the gRNA has the nucleic acid sequence
(SEQ ID NO: 1)
CCACCAGCTTACGCACACCG.

15. The method of claim 13, wherein the inhibitor of AMH is polynucleotide encoding a gRNA and Cas gene, wherein the gRNA has the nucleic acid sequence
(SEQ ID NO: 2)
GGGAGCCAACACCCTCGCTG.

16. The method of claim 13, wherein the inhibitor of TIMP1 is polynucleotide encoding a gRNA and Cas gene, wherein the gRNA has the nucleic acid sequence
(SEQ ID NO: 3)
GTAGACGAACCGGATGTCAG.

17. The method of claim 13, wherein the inhibitor of GNMT is polynucleotide encoding a gRNA and Cas gene, wherein the gRNA has the nucleic acid sequence
(SEQ ID NO: 4)
CAGCCATGCCTTGTACTCGG.
